(19) United States
(12) Patent Application Publication
(10) Pub. No.: US 2011/0022052 A1
(43) Pub. Date: Jan. 27, 2011

(54) Method and Devices for Force-Limiting Trigger Mechanism

(75) Inventor: Glen Jorgensen, Jacksonville, FL (US)

Correspondence Address: HUNTON & WILLIAMS LLP;INTELLECTUAL PROPERTY DEPARTMENT, RIVERFRONT PLAZA, EAST TOWER, 951 EAST BYRD ST., RICHMOND, VA, 23219-4074, US

(73) Assignee: OrthoDynamix, LLC

(21) Appl. No.: 12/507,325

(22) Filed: Jul. 22, 2009

(51) Int. Cl. F16C 1/12; A61B 17/32; A61B 17/56

(52) U.S. Cl. 606/83; 74/502.2; 606/174

(57) ABSTRACT

An actuating mechanism for use with a surgical instrument having a handle, an elongate body member, and a tool head. The actuating mechanism comprises a cable engagement arm pivotably attached at a first arm end. The cable engagement arm has a lever engagement portion at a second arm end. The actuating mechanism also comprises a cable engagement mechanism connecting a cable to the cable engagement arm and an actuation lever having a lever body. The actuating mechanism also comprises a torsion control mechanism having a piston slidably disposed within the piston chamber and a biasing mechanism configured for biasing the piston toward the chamber opening. The piston, the biasing mechanism, and the lever engagement portion cooperate to resist rotation of the actuation lever when a rotational force is applied to the actuation lever, but allow such rotation if the rotational force produces a moment that exceeds a predetermined limit.

### FIELD OF THE INVENTION

The present invention relates generally to devices and methods for performing arthroscopic procedures, particularly arthroscopic procedures on the hip, shoulder, and knee, including arthroscopic diagnostic and surgical procedures.

### BACKGROUND OF THE INVENTION

Arthroscopic surgery frequently requires that tissue be resected by small punch jaws that can be inserted through a small, tube-like cannula portal into the joint capsule. Several tissue types, specifically the labrum of the hip and the meniscus of the knee, are particularly difficult to punch. These punch jaws are typically connected to spring-loaded guns with trigger mechanisms that allow a user to manipulate the punch jaws.

One of the disadvantages associated with spring-loaded guns used for arthroscopic procedures is that sometimes the trigger force exceeds the spring strength. This can create a significant spring recoil once the spring gun is triggered and the punch jaws are engaged.

In addition to the recoil, the stress created in the jaws of spring-loaded guns used for arthroscopic procedures can lead to breakage of the jaws or other components. As would be expected, the working stresses in the punch jaws are necessarily high as they are required to puncture tough tissues, yet still enter the capsule through an approximately 5.0 mm tube-like cannula portal. As punch jaws wear down because of stresses incurred in carrying out their normal functions, more force is required to punch through these tough tissues. The additional punching force increases the stress in the jaws and, if enough excessive force is applied, the jaws can break. This breakage may result in the release of small and large fragments of the broken jaws as well as other pieces of the device into the joint capsule.

Broken jaws are not uncommon in arthroscopic instruments and can result in major and costly surgical procedures designed to retrieve the broken jaw fragments. Retrieval of these broken jaw fragments can also be extremely dangerous and lead to extreme pain and discomfort for the patient. Broken jaw failure is generally attributed to excessive force on the hand-piece trigger which is directly transmitted to the jaws.

### SUMMARY OF THE INVENTION

The invention generally relates to an actuating mechanism for use with a surgical instrument having a handle, an elongate body member extending distally from the handle and terminating in a tool head configured for accomplishing a surgical action, the tool head being operable by application of a force to a cable extending from the tool head to the handle. The actuating mechanism comprises a cable engagement arm pivotably attached at a first arm end to the handle by a first pivot for rotation about a first pivot axis. The cable engagement arm has a lever engagement portion at a second arm end spaced apart from the first pivot. The actuating mechanism also comprises a cable engagement mechanism connecting a cable to the cable engagement arm at a point spaced apart from the first pivot whereby rotation of the cable engagement arm causes a change in a force applied to the cable. The actuating mechanism further comprises an actuation lever having a lever body with a lever pivot end and a lever free end and a tang extending from the lever pivot end. The tang is pivotably attached to the cable engagement arm by a second pivot for rotation relative to the cable engagement arm. The lever body has a piston chamber with a chamber opening at the lever pivot end. The actuating mechanism also comprises a torsion control mechanism having a piston slidably disposed within the piston chamber and a biasing mechanism configured for biasing the piston toward the chamber opening. The piston has an arm engagement portion configured for engaging the lever engagement portion of the cable engagement arm. The piston, the biasing mechanism, and the lever engagement portion cooperate to resist rotation of the actuation lever about the second pivot when a rotational force is applied to the actuation lever, but allow such rotation if the rotational force produces a moment about the second pivot exceeding a predetermined limit.

The invention also relates to a method of performing a surgical procedure within a confined body cavity of a patient using a surgical instrument having a handle, an elongate body member extending distally from the handle and terminating in a tool head, a cable engagement arm pivotably attached to the handle by a first pivot, an actuation lever pivotably attached to the cable engagement arm by a second pivot, and a torsion control mechanism configured to resist rotation of the actuation lever about the second pivot when a rotational force is applied to the actuation lever, but to allow such rotation if the rotational force produces a moment about the second pivot exceeding a predetermined limit, the tool head being operable by application of a force to a cable extending from the tool head to the cable engagement arm. The method further comprises inserting the tool head and at least a portion of the elongate body member into the body cavity and positioning the tool head for operation at a desired location within the body cavity. The method also comprises applying a rotational force to the actuation lever to produce a first moment about the first pivot and a second moment about the second pivot, the second moment being less than the predetermined limit, thereby causing the actuation lever and the cable engagement arm to rotate about the first pivot and applying a force to the cable. Upon encountering resistance to the rotational force, the rotational force is increased so that the second moment exceeds the predetermined limit, thereby causing the actuation lever to rotate about the first pivot.

Other aspects and advantages of the invention will become apparent from the following description, taken in conjunction with the accompanying drawings, illustrating the principles of the invention by way of example only.

### BRIEF DESCRIPTION OF THE DRAWINGS

The foregoing and other objects, features, and advantages of the present invention, as well as the invention itself, will be more fully understood from the following description of various embodiments, when read together with the accompanying drawings, in which:

FIG. 1 shows a side view of a surgical instrument according to an embodiment of the invention.

FIG. 2 shows a cross-sectional detailed side view of the tool head of the device of FIG. 1.

FIG. 3 shows a cross-sectional detailed side view of a portion of an actuating mechanism according to an embodiment of the invention.

FIG. 3A shows a representation of the forces being applied to the actuating mechanism.

FIG. 4A shows a cross-sectional detailed side view of a portion of the actuating mechanism according to an embodiment of the invention.

FIG. 4B shows a cross-sectional detailed side view of a portion of the actuating mechanism according to an embodiment of the invention.

### DETAILED DESCRIPTION OF THE INVENTION

The devices and methods of the invention are primarily illustrated and described herein by means of devices which have been adapted for use in performing arthroscopic procedures primarily on, but not limited to, the hips, knees, and shoulders. The devices and methods provide access to the internal portions of the distended hip capsule during arthroscopic procedures that are presently not accessible using currently available arthroscopic instruments. The devices and methods can suitably be used to perform arthroscopic procedures not only on the hip, but also on other parts of the body, such as the knee, shoulder, wrist, elbow, etc. The devices are particularly suitable for performing procedures on parts of the body that require flexible access. The devices and methods are not limited to arthroscopy, and can further be used in endoscopic and laparoscopic procedures as well as open surgeries. As described in U.S. patent application Ser. No. 12/119,799 filed May 13, 2008 (“the '799 Application”), which is incorporated herein by reference in its entirety, the devices can be in the general form of any conventional diagnostic or operative instrument including, but not limited to, graspers, scissors, forceps, scalpels, punches, probes, dissectors, mono polar cautery, bi-polar ablation/cautery, CCD camera and lenses. Thus, the disclosure to follow should be construed as illustrative rather than in a limiting sense.

Embodiments of the invention are designed to limit the amount of force that can be applied by or to the operable end of surgical instruments. The operable end of such instruments can be in the form of graspers, scissors, forceps, scalpels, punches, probes, dissectors, mono polar cautery, bi-polar ablation/cautery, CCD cameras and lenses and are referred to herein as a “tool head” or “tool heads.” The tool head can include arms, jaws, or other elements movable with relation to each other, and the device can further include a pivot arm at its proximal end. The tool head is configured to perform a surgical action on the surgery patient. The operable end can be fabricated out of a lightweight and strong bio-compatible material. The material can be selected from surgical grade stainless steel, anodized aluminum, and polymeric materials and composites.

In many arthroscopic surgical procedures, a device's operable end is capable of resecting or punching through the tough tissue of the labrum of the hips and the meniscus of the knees. Surgical devices that use spring-loads to manipulate an operable end capable of engaging tissue sometimes fail when the force applied to the tool head exceeds the maximum safe force level. This maximum safe force level is a predetermined limit that may vary based on the type of surgery and the body part being operated on. Spring loads can sometimes fail and create a significant and potentially dangerous recoil effect, and lead to broken arms, jaws, or elements. Retrieving broken fragments can be dangerous, extremely painful, and result in costly and time-consuming follow-up surgeries.

The present methods and devices were created to limit the amount of force on the tool head of a surgical instrument. Further, the present methods and devices minimize the recoil from engaging tissue and eliminate or significantly reduce the amount of broken fragments by limiting the amount of force placed on tool heads capable of resecting or punching through tough tissue.

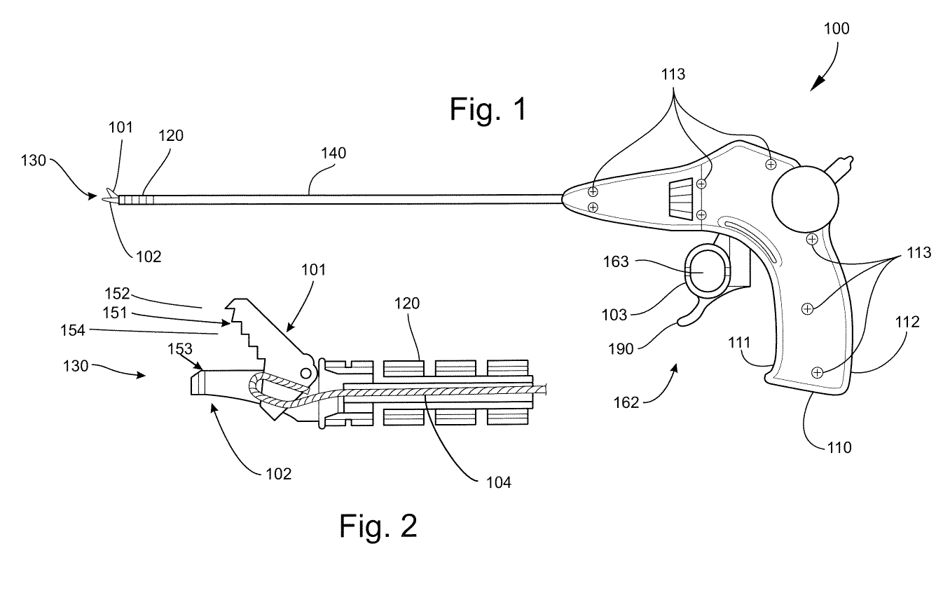

FIG. 1 illustrates one embodiment of a surgical instrument 100. The surgical instrument 100 has a proximal end 110, a distal end 120 defining a tool head 130 of the device, and an elongate body member 140 extending therebetween. As used herein, “elongate” generally refers to a member or element that is long in proportion to width, “proximal” generally refers to a position or direction that corresponds to the user, and “distal” generally refers to a position or direction that corresponds to the patient.

The elongate body member 140 is shown having a generally cylindrical shape with a circular cross-section. In an exemplary embodiment, the body member 140 includes a smooth outer surface. The elongate body member 140 is also shown having a straight, rigid shape along a substantial portion of its length. Nevertheless, this shall not be construed as limiting the body member 140 to such a shape, as it is within the scope of the present invention for other geometric shapes to be used for the elongate body member 140. For example, a flexible elongate body member 140 will have important utility in certain applications, especially as they relate to endoscopic requirements into any of the long, tortuous, cavities of the body commonly encountered especially in ENT and colorectal procedures.

The elongate body member 140 can be fabricated from any bio-compatible material known to those skilled in the art for use in fabricating medical instruments. The material can be lightweight and strong and can include, for example, surgical grade stainless steel, anodized aluminum, and polymeric materials and composites. The dimensions of the surgical instrument 100 can vary depending on the type of procedure performed and can be readily determined by one of skill in the art. In general, the length and thickness of the device is in accordance with conventional surgical instruments.

The proximal end 110 can include a handle 111 that is grasped by a user, and can be adapted to assist the user in securely gripping and manipulating the surgical instrument 100. For example, the handle 111 can include, but is not limited to, a rubber coating, grooves, or similar finger grip configuration (e.g., surface preparations or artifacts). In one preferred embodiment, the handle 111 resembles and feels like the handle of a pistol with rubber coated and notched grooves attached to the gripping surface.

The distal end 120 defines a tool head 130 of the device and can be in the form of conventional surgical and diagnostic surgical instrument operable ends. For example, the tool head 130 can be in the form of graspers, scissors, forceps, scalpels, punches, probes, dissectors, mono polar cautery, bi-polar ablation/cautery, CCD camera and lenses. The general design of the tool head 130 can be in accordance with conventional operable ends.

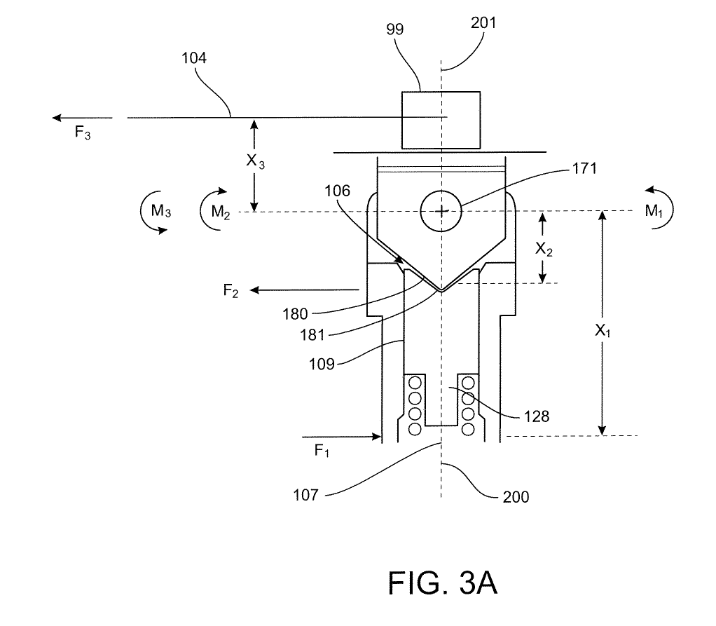

In embodiments wherein the tool head 130 is in the form of, for example, graspers or scissors, which include a pair of arms, jaws or other elements that are movable in relation to each other, the device includes an actuation lever 103 in connection with the tool head 130 and configured and arranged to move the arms, jaws, or elements of the tool head 130. In one embodiment, the handle 111 is an actuating handle that, when manipulated, moves the arms, jaws, or other elements. Such actuating handles are well known and, therefore, the present handle 111 can be in accordance with conventional actuating handles. In one embodiment, the handle 111 includes an actuation lever 103 (as shown in FIGS. 1 and 3) engaged by a finger or thumb of the user. A user, described interchangeably herein as a surgeon, manipulates the actuation lever 103 by, for example, pressing the actuation lever 103 towards the handle 111, thus causing the arms, jaws, or other elements to open or close. In some embodiments, the handle 111 can be similar to the handle of scissors or the like, with finger or thumb hole 163 that can be opened and closed to relax or tighten the arms, jaws, or other elements. In other embodiments, one or more actuating buttons (not shown) are provided that open and close the arms, jaws, or other elements when pressed.

As shown in FIG. 3, the actuation lever 103 has a tang 161 that can be either integrally formed to the body of the surgical instrument 100, or otherwise attached to the surgical instrument 100 (e.g., by welding or some other connection means). The tang 161 is attached at a lever pivot end 174 of the actuation lever 103 and extends upward from the lever pivot end 174. The actuation lever 103 also has a distal end 162, a lever body 129, and a lever free end 175. The surgeon exerts a torque on the pivot arm's distal end 162 to manipulate the tool head 130. In some embodiments of the device, the actuation lever 103 is made of semi-flexible material that allows the actuation lever 103 to bend to a certain point and then cease bending. In this embodiment, the torque exerted on the tool head 130 is limited to the amount of torque that can be exerted on the semi-flexible actuation lever 103 before it ceases to bend.

In one embodiment of the invention, the jaws 101 and 102 are mounted on top of each other and fit together when in the closed position. In some embodiments, the upper jaw 101 is movable and contains serrated metal teeth 151 and a sharpened metal tip 152, while the lower jaw 102 is fixed and sharpened on the edges that come in contact with the upper jaw. The jaws 101 and 102 in this embodiment fit together when closed, but there are some embodiments where the jaws 101 and 102 do not fit together or align when closed. In some embodiments, the upper jaw 101 is fixed and sharpened on the edges that come in contact with the lower jaw 102, while the lower jaw 102 is movable and contains serrated metal teeth and a sharpened metal tip. In some embodiments, both jaws 101 and 102 are movable and contain serrated teeth that fit together when the jaws 101 and 102 are in the closed position.

In embodiments wherein the tool head 130 has arms, jaws, or elements that are controllable by a actuation lever 103, the body member 140 can be hollow and house an apparatus that connects the actuation lever 103 to the tool head 130. Manipulation of the actuation lever 103 causes the apparatus to open and close the arms, jaws, or other elements.

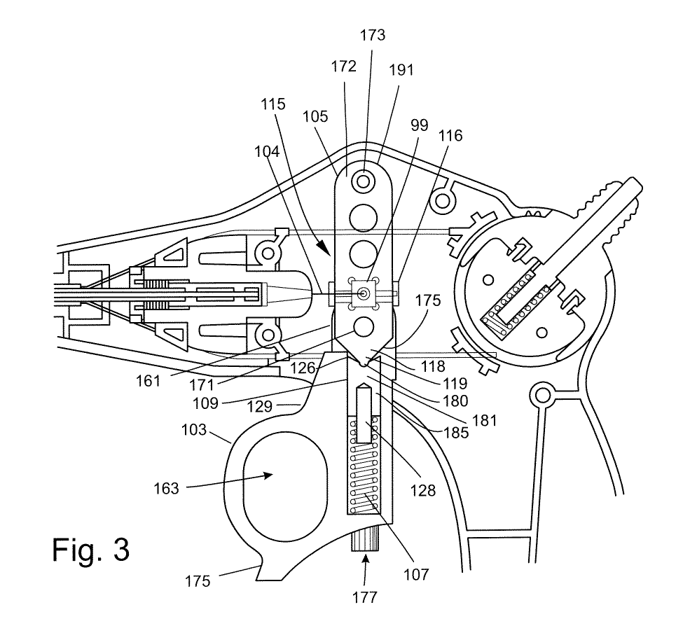

As shown in FIG. 2, the body member 140 can house one or more pull cables 104 in connection with a cable engagement arm 115. The cable engagement arm 115 is pivotably attached at a first arm end 116 by a pivot 173 to the body of the surgical instrument 100 or to the handle 111 by screws or other attachment means. The cable engagement arm 115 is attached to a lever engagement portion 118 at a second arm end 119 in the tool head 130 of the surgical instrument 100.

In one embodiment of the invention, a cable engagement mechanism 99 is connected to the cable 104 and the cable engagement arm 191 at some distance away from the pivot 173. Rotation of the cable engagement arm 191 by a force applied to the actuation lever 103 may cause an increase or decrease in the tensile force to the cable 104 and may cause the tool head 130 to open and close jaws 101 and 102. The cable engagement mechanism 99 may be a barrel swivel or other free rotational mechanical component capable of engaging the cable 104. In other embodiments, arms, jaws or similar movable or grasping mechanisms use push/pull rods in connection with the cable engagement mechanism 99 to open and close the arms, jaws, or similar movable or grasping mechanisms based on manipulation of the actuation lever 103.

One type of actuating means in the form of a actuation lever 103 for controlling the movement of the tool head 130 is shown in FIG. 1. Also, as shown in FIG. 3, the actuation lever 103 can use a cable engagement mechanism 99 to control the cable 104 and to provide support when the cable 104 is in compression. In some embodiments, the proximal end of the cable 104 is fixed to the cable engagement mechanism 99 in a manner that causes the cable 104 to be put into tension when the actuation lever 103 is pulled, and into compression when the actuation lever 103 is pushed forward or released. As shown in FIG. 2, the cable 104 is fixed in the tool head 130 in a manner that causes the tool head 130 to actuate when force is applied to the actuation lever 103 (e.g., jaws 101 and 102 close when the actuation lever 103 is pulled, and open when the actuation lever 103 is pushed forward or vice-versa). In some embodiments, the jaws 101, 102 are compressed and close when the actuation lever 103 is pushed forward, and the jaws 101, 102 open when the actuation lever 103 is released. Therefore, the invention can use a tensile force, a compressive force or both tensile and compressive forces to manipulate the jaws 101, 102.

In one embodiment of the invention, the tool head 130 can be in the form of a pair of jaws 101, 102 that, when disposed in a closed position, overlap each other to resect or punch tissue positioned between the pair of jaws 101, 102. In some embodiments, the jaws 101, 102 are mounted on top of each other and fit together when in the closed position. In some embodiments, the upper jaw 101 is movable and contains serrated metal teeth 151 and a sharpened metal tip 152, while the lower jaw 102 is fixed and sharpened on the edges 153 that come in contact with the upper jaw 101. In this embodiment, the jaws 101, 102 fit together when closed. In some embodiments the jaws 101, 102 do not fit together or align when closed. In some embodiments, the upper jaw 101 is fixed and sharpened on the edges 154 that come in contact with the lower jaw 102, while the lower jaw 102 is movable and contains serrated metal teeth and a sharpened metal tip. In some embodiments, both the upper and lower jaws 101, 102 are movable and contain serrated teeth that fit together when the jaws 101, 102 are in the closed position.

The upper jaw 101 may be actuated by a cable 104 that is connected to the cable engagement mechanism 99 and is actuated by torque being applied to the actuation lever 103. With tissue positioned between the upper and lower jaws 101, 102, the actuation lever 103 is actuated with sufficient torque to punch through or resect the tissue between the jaws 101, 102. The tissue can also be punctured or resected with the sharpened tip at the end of one or both jaws. In one aspect, the pivot 173 transmits force through a spring-loaded torsion control mechanism 185 that limits the amount of force transmitted to the jaws 101, 102. The surgical instrument 100 or one or more portions of the surgical instrument 100, such as the elongate body member 140, the tool head 130, etc., can be disposable.

The tool head 130 of the device may include other devices other than punch jaws including graspers, punches, scissors, RF ablative electrode(s), or CCD cameras with directional lenses. These devices can be controllable in five degrees of freedom as described in the '799 Application. In some embodiments, fewer than five degrees of freedom can be provided as desired. Further detailed description of the five degrees of freedom can be found in the '799 Application.

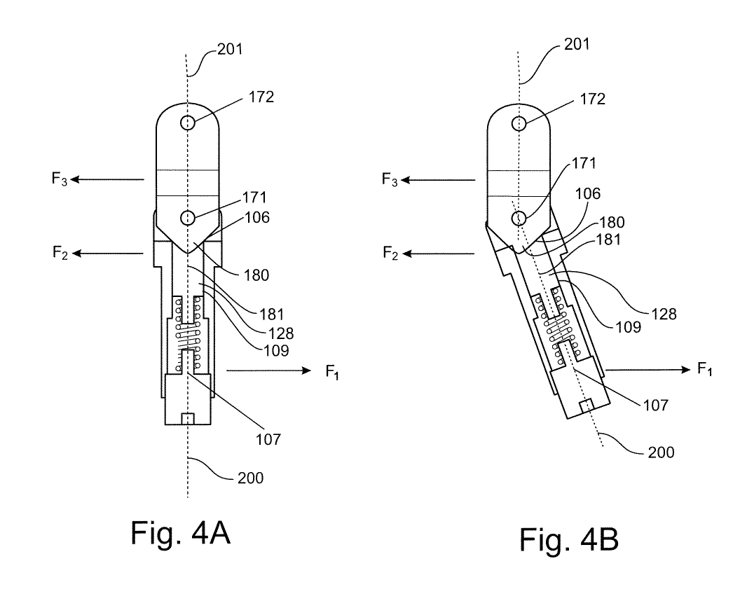

As shown in FIG. 3, to limit the amount of torque that can be applied to the actuation lever 103 to a level that will not break the jaws 101 and 102, or the cable 104, the actuation lever 103 is configured to transmit the torque through an actuating mechanism 105. As shown in FIGS. 3A and 4A-B, the torsion control mechanism 185 has a piston 128 that is inserted into a piston chamber 109. The piston 128 is kept in tension by a spring 107 that pushes the piston 128 towards the piston chamber 109. The arm engagement portion 126 is designed to engage the lever engagement portion 118 of the cable engagement arm 191, the piston 128, and the spring 107.

In some embodiments of the invention, the piston 128 has a piston chamber 109 with a small trough 181 of equal radius in a chamber opening 106 that mates with a small radius tip, or bar 180. The bar 180 can be in the shape of a wedge, cone, circle, conical depression, or other shape as long as it fits into a corresponding receptacle in the trough 181.

The amount of torque produced by a given force about a particular point depends on the distance between that point and the point of application of the force. FIG. 3A illustrates the significant force and torque relationships of the device 100. Of particular significance is the relationship of the torque produced by the force applied by the user to the resistant force and torque produced at the point where the piston chamber 109 meets the chamber opening 106, or more specifically, where the bar 180 mates with the trough 181. In the exemplary embodiment of FIG. 3A, the axis 200 of the actuation lever 103 remains substantially aligned with the axis 201 of the cable engagement arm 191 as long as the torque about the pivot 171 connecting the actuation lever 103 and the cable engagement arm 191 does not exceed a level determined by the resistance force. In the illustrated embodiment, the bar 180 has to “jump” out of the trough 181 before the actuation lever 103 can break the alignment between the actuation lever actuation lever axis 200 and the engagement arm axis 201.

The torque produced by the force applied to the actuation lever 103, is stated as:

T=r×F⊥

where T is the torque, r is the distance from the point of interest to the point of application of the force (e.g., the place on the actuation lever 103 or the additional force lever 190 where a user applies a force), and F⊥ is the component of the user-applied force perpendicular to the axis of the lever passing through the point of interest.

In the actuating mechanism 105, the cable 104 is terminated at the cable engagement mechanism 99. The actuating mechanism 105 has a cable engagement arm 191 with a first arm end 172 and a first pivot for rotation 173. The actuation lever 103 is connected to the lever engagement portion 118 at the tang 161 by a second pivot for rotation 171. The second pivot for rotation 171 also engages the cable engagement arm 191 about a pivot axis that is parallel to the pivot axis formed by the first arm end 172 and the first pivot for rotation 173, and perpendicular to the axis formed by the cable engagement arm 191.

The cable 104 can be a cable or rod that is selectively connectable to the actuation lever 103 by means of a cable engagement mechanism 99 or other attachment means. The torque applied to the actuation lever 103, however, is not directly coupled to the cable engagement mechanism 99. When a user applies a force F₁ on the actuation lever 103, a force is also produced on cable 104 in the same direction. The level of force applied to the cable is determined by (1) the distance from the first pivot 172 at which the force F₁ is applied, and (2) the distance between the first pivot 172 and the point at which the cable 104 is attached to the actuation lever 103. The force applied to the cable is balanced by a reaction force F₃ applied by the cable to the actuation lever 103. The forces F₁ and F₃ produce torques M₁ and M₃, respectively, about the second pivot point 171, the level of which is determined by their relative distances X₁ and X₃ from the second pivot point 171. As these forces are applied, a resistance force F₂ is exerted on the piston chamber 109 and the chamber opening 106, which produces a torque M₂ in the opposite direction of M₁ and M₃. As long as the resistant force stays below the level at which the bar 180 “jumps” out of the trough 181, the torques M₁ and M₃ will remain balanced with M₂:

|M₁|+M₃|=|M₂|

The actuation lever 103 is rotatably and selectively coupled to a piston chamber 109 and a chamber opening 106. The spring 107 transmits a torque on the piston chamber 109 that results in an opposing torque, M₂, as shown in FIGS. 3A. As shown in FIGS. 4A and 4B, when the torque on M₃ is greater than the predetermined torque, the piston chamber 109 rotates away from the chamber opening 106, and the actuation lever 103 rotates to a hard stop. Immediately upon release of the actuation lever 103, the piston chamber 109 aligns itself with the chamber opening 106, ready for another applied torque.

The opposing torque M₂ is proportional to the force on the spring 107 and is calibrated so that if M₂ is greater than the predetermined torque, then the actuation lever 103 rotates to a hard stop. The spring 107 is positioned to pre-load the piston 128 with a force that, under less than maximum stress, aligns the piston chamber 109 with the chamber opening 106 for the purpose of transferring torque to the cable 104. For example, if the jaws 101 and 102 are in contact with too much tissue, or the tissue type cannot be punched through, then the force to the jaws 101 and 102 may approach the 65-pound maximum safe force limit if the surgeon continues to apply greater and greater torque to the actuation lever 103. When the 65-pound maximum safe force limit is reached, the spring 107 is unable to maintain the alignment between the chamber opening 106 and the piston chamber 109, thus allowing the bar 180 to “break” out of the trough 181 and move to a hard stop as shown in FIG. 3A. This means that no matter how much additional torque M₁ is applied to the actuation lever 103, the torque M₃ on the cable 104 remains the same or decreases once the actuation lever 103 returns to the hard stop position. This limits the force on jaws 101 and 102 and cable 104 to a value equal to or less than the predetermined limit (i.e., the maximum safe level).

In one exemplary embodiment of the invention, the piston 128, the spring 107, and the lever engagement portion 118 of the cable engagement arm 191 cooperate to resist rotation of the actuation lever 103 about the second pivot 171 when the engagement arm axis and the actuation lever arm axis are aligned and a rotational force is applied to the actuation lever 103. Rotation is allowed, however, if the rotational force produces a moment about the second pivot 171 exceeding a predetermined limit.

The actuation lever 103 may come in a variety of different sizes, shapes, or configurations. In one embodiment, the actuation lever 103 resembles a trigger with an additional force lever 190 added to the actuation lever 103 for leverage. The additional force lever 190 allows the surgeon to create additional leverage on the actuation lever 103, thus creating more torque, and allowing the surgeon to conserve energy.

In one embodiment of the invention, the safe torque level will be based on the type of surgery. In some embodiments of the invention, the safe torque level is pre-determined and applicable for all devices and types of surgeries.

In one embodiment of the device, the safe torque level may be adjusted by the surgeon. By turning a screw 177, the surgeon can compress or de-compress the spring 107, thus increasing or decreasing the amount of torque a surgeon can apply before the bar 180 jumps out of the trough 181. In this embodiment, the surgeon can increase and decrease the safe torque level applied to the tool head 130.

As shown in FIGS. 3A and 4A-B, the chamber opening 106 is above the piston chamber 109. In some embodiments of the device, however, the position of the piston chamber 109 and the chamber opening 106 are reversed and the chamber opening 106 is below the piston chamber 109.

As shown in FIG. 4A, the bar 180 and the trough 181 are in axial alignment as no torque has been exerted on the actuation lever 103. As shown in FIG. 4B, as force F₁ is exerted on the actuation lever 103, the axial alignment between the bar 180 and the trough 181 is broken, and the bar 180 moves along the trough 181 to the left edge of the chamber opening 106.

For all of the embodiments, all or portions of the device can be reusable or disposed of. In some embodiments, removable and interchangeable distal ends, inner/outer body member(s), and/or elongate body members that can be reused or disposed of as desired.

In another aspect, the invention generally relates to a surgical instrument kit, comprising one or more of the components set forth herein. The one or more devices can be packaged in sterile condition.

### Testing

According to a design review of the invention, force measurements found that the jaw closing force, F₁, can range from 9 to 22 pounds depending on the sharpness of the jaws 101 and 102 and the amount of tissue in the jaws 101 and 102. The force in the cable 104 to effect the forces on jaws 101 and 102 may be as high as 42 pounds. Therefore, taking into account losses and the additive effect of the spring 107, 65 pounds was chosen as the maximum force on the jaws 101 and 102.

The peak stress level in the jaws 101 and 102 is then 117K psi as estimated by the SolidWorks stress analysis program. The 5% yield value of the 17−4 stainless H-900 is 166K psi and the ultimate stress is 188K. Working at a factor of safety less than 2 requires unusual measures to protect the jaws 101 and 102 against any actuating forces greater than 65 pounds.

The tests found that the amount of force reduction is a function of how far the piston chamber 109 travels before coming in contact with the hard stop. Once the maximum force on the actuation lever 103 is reached, the actuating mechanism 105 will move forward slightly and reduce the force on the cable 104. The force on the cable 104, however, is still sufficient to retain a grip on the tissue in the jaws 101 and 102.

The tests also found that if the trough 181 for the bar 180 is deeper, then the preload force to prevent it from jumping out of the trough 181 decreases. Therefore, with relatively low preload forces, it is possible that relatively high break-away forces can be generated. If, on the other hand, the trough 181 is shallow, then it takes high preload forces to hold the vertical axial alignment.

Methods of the present invention comprise performing arthroscopic procedures using the present devices. During use, the handle 111 or proximal end 110 is positioned outside the body. At least the distal portion of the body member is positioned inside the joint capsule. In one embodiment, two incisions are made and a cannula is inserted through each incisions to provide access to the joint capsule. The elongate body member 140 of one surgical instrument 100 having a visualization mechanism at its distal end 120 is inserted through one cannula. The elongate body member 140 of another surgical instrument 100 having a tool head 130 (e.g., scissors, dissector, forceps, punch jaws, etc.) is inserted through the other cannula. The elongate body member 140 of one or more of the surgical instruments 100 are extended and provided in a curved profile to enhance access to the various parts of the joint. In one embodiment, the body member is provided as an inner and outer body member, and, once the outer body member is positioned within the joint capsule, the inner body member is extended outside of the outer body member and provided in a curved profile. The procedure is performed and the devices withdrawn through the cannula after they are returned to a straight profile. Such procedures can be used in any type of arthroscopic surgery, such as the hip, knee, or shoulder.

In one embodiment, the invention generally relates to a method for performing minimally invasive arthroscopic surgical procedures by providing a surgical instrument 100 comprising a handle 111 with an actuation lever 103 at a proximal end 110, a flexible or curvable portion with operable devices at the distal end 120, and an elongate body member 140 extending therebetween. A tool head 130 is further rotatably mounted at the distal end 120. Further information about the bend radius of the flexible or curvable portion of some embodiments of the invention can be found in the '799 Application. The method further comprises (1) inserting the straight elongate member into the hip, knee, or shoulder capsule; (2) performing the intended procedure by actuating the operable end by, for example, tensioning a cable to the desired effect through the manipulation of control mechanisms in the handle; (3) removing the surgical instrument 100 from the body.

The present invention also includes kits (not shown) that comprise one or more devices in accordance with the invention, that can be packaged in sterile condition. Such kits also may include one or more interchangeable distal ends 120, tool heads 130, body members (e.g., an elongate body member 140) for use with the surgical instrument 100, and/or written instructions for use of the surgical instrument(s) 100 and/or the equipment. In some embodiments, the kit also can also include flexible and/or rigid access cannulas that are sealed against the saline distension pressure within the joint capsule and inserted using “safe access” trocars, mechanical flexation device(s) that mechanically distends the hip joint laterally as well as longitudinally along the line of action coincident with the center line of the femoral neck, and fluid management systems to control the flow and pressure of the saline in the hip capsule.

In one embodiment, the kit includes some combination of the following equipment: a curvilinear visualization device, a curvilinear instrument capable of mechanically manipulating tissue, such as a grasper, a punch, scissors, a clamp, a retractor, a powered instrument blade, a bone resection tool, or the like, and a curvilinear instrument capable of electrically manipulating tissue, such as a monopolar or bi-polar cautery, or the like. The visualization device, mechanical manipulating device, and electrical manipulating device can be provided as two or more proximal ends 110 or handles 111 together with interchangeable body members having thereon a variety of visualization, mechanical, and electrical elements. In some embodiments, the visualization device, mechanical manipulating device and electrical manipulating device can be provided as two or more proximal ends 110 or handles 111 with attached body members together with interchangeable inner tubular members having thereon a variety of visualization, mechanical, and electrical elements. In some embodiments, the visualization device, mechanical manipulating device and electrical manipulating device can be provided as two or more proximal ends 110 or handles 111 with attached body members together with interchangeable distal operable ends in the form of a variety of visualization, mechanical and electrical operable elements.

The foregoing description of the invention is merely illustrative thereof, and it is understood that variations and modifications can be effected without departing from the scope or spirit of the invention as set forth in the following claims. For example, the invention also has great utility beyond hip applications described herein, (e.g., knee and shoulder arthroscopy, as well as smaller joint arthroscopy). The smaller diameters of the device (e.g., approximately 3.5 mm for graspers and punch jaws) as well as the flexibility of each device also make it useful for other applications that require delicate tissue manipulation, including, but not limited to, laparoscopic cholecystectomies, appendectomies, hernia repair, bariatric gastric by-pass, and certain thoracic and spinal procedures.

What is claimed is:

1. An actuating mechanism for use with a surgical instrument having a handle, an elongate body member extending distally from the handle and terminating in a tool head configured for accomplishing a surgical action, the tool head being operable by application of a force to a cable extending from the tool head to the handle, the actuating mechanism comprising:
a cable engagement arm pivotably attached at a first arm end to the handle by a first pivot for rotation about a first pivot axis, the cable engagement arm having a lever engagement portion at a second arm end spaced apart from the first pivot;
a cable engagement mechanism connecting a cable to the cable engagement arm at a point spaced apart from the first pivot whereby rotation of the cable engagement arm causes a change in a force applied to the cable;
an actuation lever having a lever body with a lever pivot end and a lever free end and a tang extending from the lever pivot end, the tang being pivotably attached to the cable engagement arm by a second pivot for rotation relative to the cable engagement arm, the lever body having formed therein a piston chamber with a chamber opening at the lever pivot end; and
a torsion control mechanism having a piston slidably disposed within the piston chamber and a biasing mechanism configured for biasing the piston toward the chamber opening, the piston having an arm engagement portion configured for engaging the lever engagement portion of the cable engagement arm, wherein the piston, the biasing mechanism, and the lever engagement portion cooperate to resist rotation of the actuation lever about the second pivot when a rotational force is applied to the actuation lever, but allow such rotation if the rotational force produces a moment about the second pivot exceeding a predetermined limit.

2. The actuating mechanism of claim 1, wherein the predetermined limit is established based on operational limits for the tool head.

3. The actuating mechanism of claim 1, wherein the predetermined limit is established by the user.

4. The actuating mechanism of claim 1, wherein the arm engagement portion comprises an indented engagement surface and the lever engagement portion comprises a tapered sub-portion, the indented engagement surface being configured for receiving at least a portion of the tapered sub-portion therein.

5. The actuating mechanism of claim 4, wherein the tapered sub-portion terminates in a rounded knob and a base portion of the depression is configured to receive the rounded knob.

6. The actuating mechanism of claim 1, wherein the cable engagement arm is further defined by an engagement arm axis perpendicular to the first pivot axis, with the tang being pivotably attached to the cable engagement arm by the second pivot for rotation relative to the cable engagement arm about a second pivot axis parallel to the first pivot axis, and the actuation lever is further defined by an actuation lever axis, and wherein the arm engagement portion is configured to engage the lever engagement portion of the cable engagement arm, the piston, the biasing mechanism, and the lever engagement portion and cooperate to resist rotation of the actuation lever about the second pivot axis when the engagement arm axis and the actuation lever axis are aligned.

7. The actuating mechanism of claim 1, wherein the tool head comprises a set of mechanical devices capable of resecting or punching through body tissue.

8. The actuating mechanism of claim 7, wherein the set of mechanical devices is a pair of movable jaws.

9. The actuating mechanism of claim 1, further comprising:
a flexible distal end segment extending from the distal end of the outer body member, the flexible distal end segment having an axial end segment passage formed therethrough; and
a rotation control member comprising:
an extension tube portion rotatably disposed within an elongate body member; and
a flexible drive shaft portion attached to and extending distally from a distal end of the extension tube portion for rotation therewith, at least a portion of the flexible drive shaft portion being rotatably and slidably disposed within an axial end segment passage so as to take on a profile of the flexible distal end segment.

10. The actuating mechanism of claim 9, wherein the extension tube portion is operably connected to a rotation control means housed in the handle for selectively rotating the extension tube portion, the flexible drive shaft portion and the operable end while the flexible distal end segment remains fixed.

11. The actuating mechanism of claim 1, wherein the tool head comprises a plurality of vertebrae.

12. The actuating mechanism of claim 11, wherein the vertebrae are interconnected by an integral web.

13. The actuating mechanism of claim 12, wherein the vertebrae and web are integrally formed as a single member.

14. The actuating mechanism of claim 1, wherein the piston chamber is a wedge-shaped cylinder that fits into and aligns with the chamber opening.

15. The actuating mechanism of claim 14, wherein the wedge-shaped cylinder takes the form of cones, circles, bars or conical depressions that fit into and align with the chamber opening.

16. The actuating mechanism of claim 14, wherein the chamber opening takes the form of a trough.

17. The actuating mechanism of claim 1, wherein the rotation of the cable engagement arm causes a change in a tensile force applied to the cable.

18. The actuating mechanism of claim 1, wherein the rotation of the cable engagement arm causes a change in a compressive force applied to the cable.

19. A method of performing a surgical procedure within a confined body cavity of a patient using a surgical instrument having a handle, an elongate body member extending distally from the handle and terminating in a tool head, a cable engagement arm pivotably attached to the handle by a first pivot, an actuation lever pivotably attached to the cable engagement arm by a second pivot, and a torsion control mechanism configured to resist rotation of the actuation lever about the second pivot when a rotational force is applied to the actuation lever, but to allow such rotation if the rotational force produces a moment about the second pivot exceeding a predetermined limit, the tool head being operable by application of a force to a cable extending from the tool head to the cable engagement arm, the method comprising:
inserting the tool head and at least a portion of the elongate body member into the body cavity;
positioning the tool head for operation at a desired location within the body cavity;
applying a rotational force to the actuation lever to produce a first moment about the first pivot and a second moment about the second pivot, the second moment being less than the predetermined limit, thereby causing the actuation lever and the cable engagement arm to rotate about the first pivot and applying a force to the cable; and
upon encountering resistance to the rotational force, increasing the rotational force so that the second moment exceeds the predetermined limit, thereby causing the actuation lever to rotate about the second pivot.
